(19) United States
(12) Patent Application Publication
(10) Pub. No.: US 2024/0058490 A1
(43) Pub. Date: Feb. 22, 2024

(54) WHEEL-MOUNTED DISINFECTION SYSTEM

(71) Applicant: GENTEX CORPORATION, Zeeland, MI (US)

(72) Inventors: Thomas S. Wright, Holland, MI (US); Steven L. Geerlings, Holland, MI (US)

(73) Assignee: GENTEX CORPORATION, Zeeland, MI (US)

(21) Appl. No.: 18/333,072

(22) Filed: Jun. 12, 2023

Related U.S. Application Data
(60) Provisional application No. 63/371,676, filed on Aug. 17, 2022.

(51) Int. Cl. A61L 2/10; A61B 50/13

(52) U.S. Cl. CPC A61L 2/10; A61B 50/13

(57) ABSTRACT

According to one aspect of the present disclosure, a medical cart is disclosed. The medical cart may comprise a frame, one or more wheels, and one or more wheel disinfection assembly. The frame may be configured to support medical equipment. The one or more wheels may be mounted to the frame. The one or more wheel disinfection assembly may each be associated with one of the one or more wheels. A wheel disinfection assembly may have one or more light sources configured to emit germicidal light onto at least part of the respective wheel. In some embodiments, the may also be configured to emit the germicidal light onto a surface on which the wheels are rolled.

### CROSS REFERENCE TO RELATED APPLICATIONS

This application claims priority under 35 U.S.C. § 119(e) to U.S. Provisional Application No. 63/371,676 filed on Aug. 17, 2022, entitled “Wheel-Mounted Disinfection System for Medical Facilities,” the disclosure of which is hereby incorporated by reference in its entirety.

### TECHNOLOGICAL FIELD

The present invention generally relates to a disinfection system using a germicidal light, such as ultraviolet-C (UVC) light, and more particularly, to a disinfection system using a germicidal light to disinfect wheels on a medical cart.

### SUMMARY OF THE DISCLOSURE

The present disclosure is directed to various aspects of a disinfection system. This disinfection system may use germicidal light in order to substantially reduce or eliminate problems associated with cleaning and/or spreading bacteria and viruses by wheeled carts, particularly in a medical setting.

According to one aspect of the present disclosure, a medical cart is disclosed. The medical cart may comprise a frame, one or more wheels, and one or more wheel disinfection assembly. The frame may be configured to support medical equipment. The one or more wheels may be mounted to the frame. The one or more wheel disinfection assembly may each be associated with one of the one or more wheels. A wheel disinfection assembly may have one or more light sources configured to emit germicidal light onto at least part of the respective wheel. In some embodiments, the light sources may also be configured to emit the germicidal light onto a surface on which the wheels are rolled.

In some embodiments, the disinfection assembly may further include a housing. The housing may: at least in part, house the one or more light sources; and form a partially enclosed slot. Additionally, the wheel may be partially enclosed by the slot. In such an embodiment, the germicidal light may be emitted into at least part of the slot.

In some embodiments, the at least one of the wheels is mounted to the frame via a caster. The caster may allow the wheel to swivel respective to the frame. Additionally, the wheel disinfection assembly may be secured to the caster. In some such embodiments, the disinfection assembly may snap fit into place over at least part of the caster. In some such embodiments, the caster may have a mounting plate, an axel, one or more legs, and a swivel. The mounting plate may secure the caster to the frame. The axel may support the wheel and allowing the wheel to rotate thereupon. Additionally, the axel may extend from the leg. The swivel joint may be attached to the leg and the mounting plate allowing the leg to rotate relative to the frame.

In some embodiments, the wheel disinfection assembly may fit over one or more leg of the caster such that the legs are substantially disposed within the slot. In some such embodiments, the wheel disinfection assembly may further include a caster interface on each side of the slot. Further, the caster interfaces may secure the wheel disinfection assembly to the caster. In some embodiments, the caster interfaces may snap fit to the caster. Further, the caster interfaces may fit to the caster in alignment with the axel.

In some embodiments, the medical cart may further comprise a rechargeable battery and a generator. The rechargeable battery may power the one or more light sources. The generator may engage the wheel and generates power in response to rotation of the wheel. Further, the power generated by the generator may recharge the battery.

In some embodiments, the medical cart may further comprise a location tracking device. The location tracking device may provide real time location data of the medical cart to a remote location.

In some embodiments, the medical cart may further comprise a cart tip sensor. The cart tip sensor may detect if the cart is tipped from its normal upright position. In some such embodiments, upon detection that the cart is tipped, the light sources may be deactivated. In some embodiments, the tip sensor may include at least one of an orientation sensor and an accelerometer. In some embodiments, the tip sensor may include a light sensor. The light sensor may detect ambient light. Additionally, the tip may be detected based, at least in part, on the ambient light being detected by the tip sensor in excess of a threshold.

In some embodiments, the medical cart may further comprise a motion sensor. The motion sensor may detect the cart being in motion. Additionally, the light sources may be activated based, at least in part, on the cart being in motion, and deactivated based, at least in part, on the cart being stationary.

In accordance with another aspect of the present disclosure, a wheel disinfection assembly is disclosed. The wheel disinfection assembly may comprise a housing and a light source. The housing may form a partially enclosed slot. The light source may be at least partially enclosed by the housing. Further, the light source may be configured to emit germicidal light into the slot. Additionally, the housing may be configured to receive a wheel caster, at least partially, into the slot such that a wheel mounted to the caster is at least partially enclosed by the slot and the germicidal light is emitted onto the wheel. In some embodiments, the housing may include a caster interface on each side of the slot. The caster interfaces may be configured to snap fit to the caster to secure the wheel disinfection assembly to the caster.

These and other aspects, objects, and features of the present disclosure will be understood and appreciated by those skilled in the art upon studying the following specification, claims, and appended drawings. It will also be understood that features of each embodiment disclosed herein may be used in conjunction with, or as a replacement for, features in other embodiments.

### BRIEF DESCRIPTION OF THE DRAWINGS

In the drawings:

FIG. 1 is a perspective view of a medical cart;

FIG. 2 is a close up view of a wheels for a cart with a disinfection system;

FIG. 3 is a first view of a part of a wheel disinfection system light assembly;

FIG. 4 is a second view of a part of a wheel disinfection system light assembly; and

FIG. 5 is a block diagram schematic of a cart wheel disinfection system.

### DETAILED DESCRIPTION

The present illustrated embodiments reside primarily in combinations of apparatuses, systems, and related to a wheel disinfection system. Specifically, UV light may be used to disinfect wheels on a medical cart. Accordingly, the components of the apparatuses, systems, and methods have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

It is to be understood that the specific devices illustrated in the attached drawings and described in the following specification are simply exemplary embodiments of the inventive concepts defined in the appended claims. Hence, specific characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, apparatus, or system that comprises a list of elements does not include only those elements, but may include other elements not expressly listed or inherent to such process, method, article, apparatus, or system. An element proceeded by “comprises a . . . ” does not, without more constraints, preclude the existence of additional identical elements in the process, method, article, apparatus, or system that comprises the element.

The present disclosure is directed to a cart-wheel disinfection system 50. The cart-wheel disinfection system 50 may include a cart 10 and a wheel disinfection assembly 40.

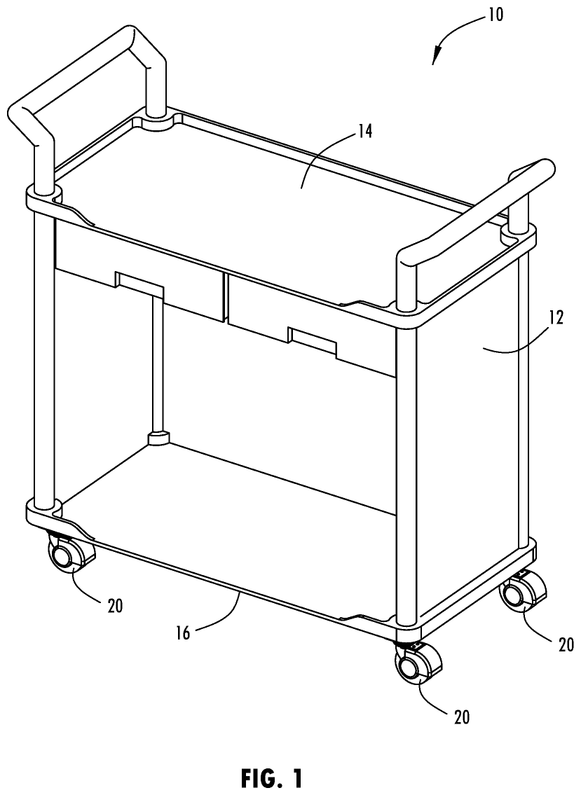

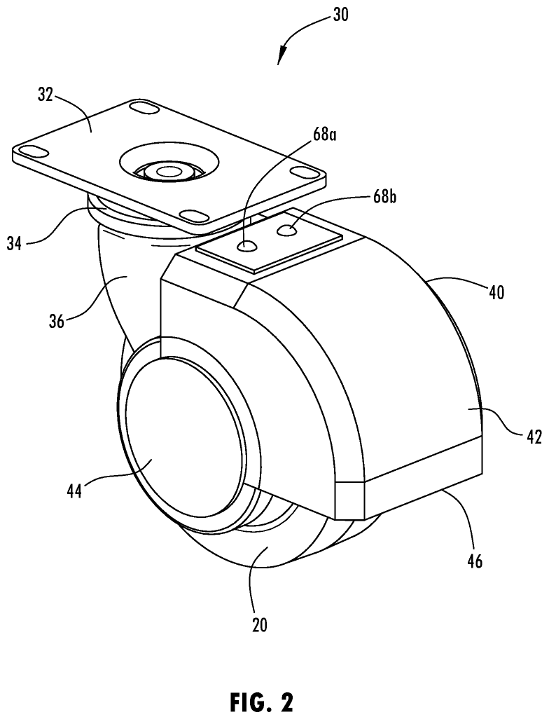

Referring to FIG. 1, Cart 10 may be a medical cart. Although the cart 10 is shown as having a couple of shelves, the term medical cart as used herein, shall mean any devices having wheels, including IV poles, tables, and beds. Additionally, cart 10 may be fit with a disinfection system. Further, cart 10 may include a frame 12, support surface 14, a bottom surface 16, and/or a plurality of wheels 20. Support surface 14 and bottom surface 16 are mounted to frame 12 and may support one or more medical instruments or pieces of equipment. Additionally, one or more of wheels 20 may be mounted to frame 12 via a caster 30 (FIG. 2).

As shown in FIG. 2, caster 30 may include a mounting plate 32, a swivel joint 34, one or more legs 36, and/or an axel (not shown). Further, caster 30 may serve to rotationally mount a wheel 20 to the cart 10. The mounting plate 32 may mount the caster 30 to the frame 12 of cart 10. The swivel joint 34 is attached to the mounting plate 32. The one or more legs 36 are attached to the swivel joint 34. Further, legs 36 may extend downward relative to the mounting plate 32. Additionally, the swivel joint 34 allows legs 36 to swivel relative to the mounting plate 32 and the frame 12. The one or more legs 36 may support the axel, which may extend from a leg 36 or between one or more legs 36. The wheel 20 is mounted on the axel about which it may rotate, relative the frame 12. In some embodiments, the axel may extend between two legs 36, and thus the wheel 20 may be disposed between two legs 36. In other embodiments, the axel may extend from a leg 36. In some such embodiments, wheel 20 may thus be disposed beside the leg 36.

Additionally, as shown in FIGS. 2-4, the cart 10 may include a wheel disinfection assembly 40. Wheel disinfection assembly may include a housing 42 and/or one or more light sources 45. The light sources 45, for example, may be light emitting diodes (LEDs). The housing 42 may, at least in part, house and/or support the one or more light sources 45. Light sources 45 may be configured to emit germicidal light. The germicidal light may be ultraviolet-C (UVC) light or any other form of light known to kill germs. Further, the one or more light sources 45 may be configured to emit light onto at least part of a wheel 20. Additionally, the one more light sources 45 may be further configured to emit light towards the ground proximate the wheel 20. As such, the wheel disinfection assembly 40 may additionally provide disinfection to the ground.

In some embodiments, the housing 42 may form and/or partially enclose a slot 48. The germicidal light may be emitted into at least part of the slot 48. Further, the wheel 20 may be disposed, at least partially, within the slot 48. Accordingly, the wheel 20 may be disinfected by the germicidal light.

In some embodiments, the wheel disinfection assembly 40 may be secured to caster 30. For example, the wheel disinfection assembly 40 may fit over the legs 36 of the caster 30. As such, the housing 42 may be secured to one or more of the one or more of the legs 36. Accordingly, the one or more legs 36 may be disposed, at least in part, within the slot 48.

In some embodiments, the one or more light sources 45 may be mounted inside a front edge 46 of the housing 42. Further, these light sources 45 may are be disposed and/or substantially enclosed by slot 48. In some embodiments, the one or more light sources 45 may be mounted inside housing 42 further within slot 48, such that it is disposed above the wheel 20 and/or in between the legs 36.

In some embodiments, the housing 42 of wheel disinfection assembly 40 may include a caster interface 44 on each side of the slot 48. As such, in some embodiments, the slot 48 may be at least partially defined by the caster interfaces 44. The caster interfaces 44 may be by which the housing 42 interfaces with or is secured to the caster 30. Further, the caster interfaces 44 may secure to caster 30 are about each end of the axel of the caster 30. In some embodiments, the caster interface 44 may be universal in that they engage multiple caster types.

In some embodiments, the wheel disinfection assembly 40 may be snap into place over at least part of a pre-existing caster 30 such that a cart 10 may be retrofit with the disinfectant system. In such an embodiment, the caster interfaces 44 of housing 42 may flex to fit around a part of caster 30, such as a part of legs 36, and then recoil around a narrower portion to secure the wheel disinfection assembly 40 to caster 30. In some embodiments, housing 42 may recoil at the axel of caster 30.

The wheel disinfection assembly 40 may have two form factors: easily retrofittable to existing carts/casters or an entirely self-contained caster design.

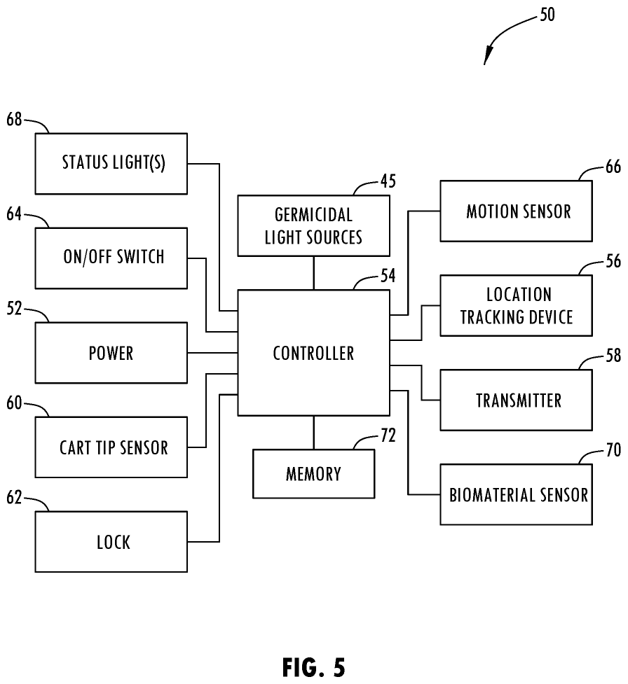

FIG. 5 shows a block diagram schematic of a cart-wheel disinfection system 50. This schematic may be enabled by one or more electrical circuits. This schematic provides for various components of the cart-wheel disinfection system 50. Each of the components may be provided for as a part of the cart 10, the caster 30, and/or the wheel disinfection assembly 40.

The cart-wheel disinfection system 50 may also include a power supply 52. The power supply 52 may provide power to the cart-wheel disinfection system 50. In some embodiments, the power supply 52 may be a battery. For example, the power supply 52 may be a sealed battery, a replaceable battery, and/or a rechargeable battery. In some embodiments the rechargeable battery may be charged by plugging it in. In some embodiments, the power supply 52 may provide power from or recharge the battery from movement of the wheel 20. In such an embodiment, power for powering and/or recharging may be enabled by a generator may be configured to engage the wheel 20 and generate power in response to rotation of the wheel 20. In some embodiments, the power supply 52 may provide power from or recharge the battery from light. In such an embodiment, the power for powering and/or recharging may be enabled by a solar panel, which provides power based on received light.

The cart-wheel disinfection system 50 may also include a controller 54. The controller 54 may operate some optional advanced features of the cart-wheel disinfection system 50. The controller 54 may comprise a memory and a processor. The memory and processor may be communicatively coupled to one another. The processor, for example, may be any device capable of processing electronic instructions. Examples include a microprocessor and/or an application specific integrated circuit (ASIC). Further, the processor may be configured to execute one or more instructions stored in the memory. The memory may be a non-transitory computer usable or readable medium, which may include one or more storage devices or articles. Accordingly, the memory may be operable to store one or more instructions. Examples of the memory include conventional hard disks, solid-state memories, random access memories (RAM), read-only memory (ROM), erasable programmable read-only memory (EPROM), electronically erasable programmable read-only memory (EEPROM), optical or magnetic disks, dynamic random access memory (DRAM).

In some embodiments, the cart-wheel disinfection system 50 may include a location tracking device 56. The location tracking device 56 may provide for facility mapping or cart location (Real Time Location Services (RTLS)). In some such embodiments, the cart-wheel disinfection system 50 may further include a transmitter 58 for transmitting the location data to a remote central location.

The cart-wheel disinfection system 50 may alternatively or additionally include a cart tip sensor 60. The cart tip sensor 60 may be configured to detect that the cart 10 has tipped from its normal upright position. Upon detection that the cart 10 has tipped, the light sources 45 may be deactivated. In some embodiments, the controller 54 may disrupt the supply of power to the light sources 45 based, at least in part, on the tip sensor 60 detecting the tip. This is an advantageous safety feature, because the germicidal light emitted from the light sources 45 may be harmful to humans under some circumstances. Although the housing 42 may act as a shroud to shield the light from striking a person, if the cart 10 is tipped, the light could otherwise project upward toward a person.

In some embodiments, the tip sensor 60 may include a light sensor, such as an ambient light sensor. The light sensor may detect ambient light. Additionally, the light sensor may face downward within the housing 42. Thus, the light sensor may detect ambient light within the slot 48 The light sensor may sense an increase in brightness of light as a result of being tipped as more ambient light may enter the slot 48. The detected increase in brightness may surpass a threshold, indicating the possibility of a tip. As such, the tip sensor 60 may detect a tip due to an increase in the sensed light from the light sensor surpassing a threshold.

In other embodiments, the tip sensor 60 may include an orientation sensor or accelerometer. As such, the tip sensor 60 may include a movable ball or the like within a tube where a change in the position of the ball from the bottom of the tube is detected.

In some embodiments, the cart-wheel disinfection system 50 may be configured to ensure the light sources 45 are not activated until after proper installation in a caster using an interlock 62. The interlock 62 may be mechanical or sensor based (e.g., magnetic, or proximity etc.). accordingly, the interlock 62 may be configured to detect whether the wheel disinfection assembly 40 is fit to the caster 30. As such, if the caster light assembly is off of the cart 10, the light sources 45 may be turned off, protecting people from exposure to the germicidal light, particularly when the wheel disinfection assembly is turned over.

If a generator is used as the power supply 52, the light sources 45 may only be activated when the wheels 20 of the cart are turning and would be deactivated when the wheels 20 are not turning. Alternatively or additionally, an optional on/off switch 64 may be provided to cut power to the light sources 45. Additionally or alternatively, a motion sensor 66 could alternatively be provided for sensing movement of the wheel 20. In such an embodiment, when the cart 10 is stationary, the circuit 50 may go into a low power sleep mode where the light sources 45 are deactivated. Conversely, the light sources 45 may be activated based, at least in part, on the cart 10 being in motion.

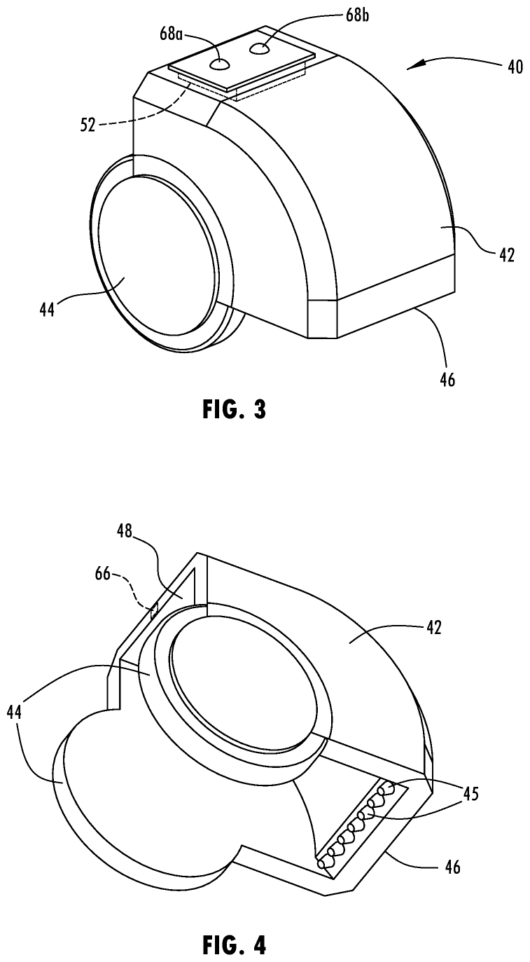

In some embodiments, the cart-wheel disinfection system 50 may include one or more status lights 68 (shown as 68a and 68b in FIG. 3). The one or more status lights 68 may indicate a system status or status of the light sources 45 and/or wheel disinfection assembly 40. For example, the status lights 68 may indicate various conditions such as a low battery 68a, active sterilization 68b when the lights 45 are activated, charging, maintenance needed, UVC dosage needed, etc.

In some embodiments, the cart-wheel disinfection system 50 may include a biomaterial sensor 70. The biomaterial sensor 70 may be provided in the housing 42. Further, the biomaterial sensor 70 may detect the presence of germs or to identify specific detected germs. In some embodiments, the output of the sensor 70 could be provided to the controller 54, which could activate the lights sources 45 when germs or other biomaterial are sensed.

In some embodiments, the controller 54 may include a memory 72 for storing data such as maintenance notices, disinfection log, cleaning effectiveness, biomaterial sensing data, and/or data from general environmental sensors (e.g., vapor sensing), etc. Such data may be transmitted to a remote central location via the transmitter 58.

A major benefit provided by the disinfecting system is limiting the transfer of contaminants between rooms and locations within the hospital. Carts, even clean carts on wheels, are rolled all over the place and cleaning the wheels could greatly reduce the spread of infections.

Although described with respect to a medical cart, the system described above may be mounted on a post (like a bottom of an IV pole) or the bottom of a cart to clean the floor. Further, it could be connected by wire to casters, which could generate power for the battery.

It will be understood by one having ordinary skill in the art that construction of the described invention and other components is not limited to any specific material. Other exemplary embodiments of the invention disclosed herein may be formed from a wide variety of materials, unless described otherwise herein.

As used herein, the term “and/or,” when used in a list of two or more items, means that any one of the listed items can be employed by itself, or any combination of the two or more of the listed items can be employed. For example, if a composition is described as containing components A, B, and/or C, the composition can contain A alone; B alone; C alone; A and B in combination; A and C in combination; B and C in combination; or A, B, and C in combination.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the invention as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

It will be understood that any described processes or steps within described processes may be combined with other disclosed processes or steps to form structures within the scope of the present invention. The exemplary structures and processes disclosed herein are for illustrative purposes and are not to be construed as limiting.

It is also to be understood that variations and modifications can be made on the aforementioned structures and methods without departing from the concepts of the present invention, and further it is to be understood that such concepts are intended to be covered by the following claims unless these claims by their language expressly state otherwise.

What is claimed is:

1. A medical cart, comprising:
a frame configured to support medical equipment;
one or more wheels mounted to the frame;
one or more wheel disinfection assembly, each associated with one of the one or more wheels, a wheel disinfection assembly having one or more light sources configured to emit germicidal light onto at least part of the respective wheel.

2. The medical cart of claim 1, wherein the one or more lights sources are also configured to emit the germicidal light onto a surface on which the wheels are rolled.

3. The medical cart of claim 1, wherein the disinfection assembly further includes a housing, wherein:
the housing:
at least in part, houses the one or more light sources; and
forms a partially enclosed slot, and
the wheel is partially enclosed by the slot.

4. The medical cart of claim 3, wherein the germicidal light is emitted into at least part of the slot.

5. The medical cart of claim 3, wherein:
at least one of the wheels is mounted to the frame via a caster;
the caster allows the wheel to swivel respective to the frame; and
the wheel disinfection assembly is secured to the caster.

6. The medical cart of claim 5, wherein the disinfection assembly is snap fit into place over at least part of the caster.

7. The medical cart of claim 5, wherein:
the caster has:
a mounting plate securing the caster to the frame,
an axel supporting the wheel and allowing the wheel to rotate thereupon;
a leg from which the axel extends; and
a swivel joint attached to the leg and the mounting plate allowing the leg to rotate relative to the frame.

8. The medical cart of claim 7, wherein the wheel disinfection assembly fits over the leg of the caster such that it is substantially disposed within the slot.

9. The medical cart of 8, wherein the wheel disinfection assembly further includes a caster interface on each side of the slot, the caster interfaces securing the wheel disinfection assembly to the caster.

10. The medical cart of claim 9, wherein the caster interfaces snap fit to the caster.

11. The medical cart of claim 9, wherein the caster interfaces fit to the caster in alignment with the axel.

12. The medical cart of claim 1, further comprising:
a rechargeable battery that powers the one or more light sources; and
a generator engaging the wheel that generates power in response to rotation of the wheel;
wherein the power generated by the generator recharges the battery.

13. The medical cart of claim 1, further comprising: a location tracking device providing real time location data to a remote location.

14. The medical cart of claim 1, further comprising: a cart tip sensor to detect if the cart is tipped from its normal upright position.

15. The medical cart of claim 14, wherein: upon detection that the cart is tipped, the light sources are deactivated.

16. The medical cart of claim 14, wherein the tip sensor includes at least one of an orientation sensor and an accelerometer.

17. The medical cart of claim 14, wherein:
the tip sensor includes a light sensor to detect ambient light within the slot; and
a tip is detected based, at least in part, on ambient light being detected by the tip sensor in excess of a threshold.

18. The medical cart of claim 1, further comprising:
a motion sensor to detect the cart being in motion;
wherein the light sources are:
activated based, at least in part, on the cart being in motion, and
deactivated based, at least in part, on the cart being stationary.

19. A wheel disinfection assembly, comprising:
a housing forming a partially enclosed slot;
a light source at least partially enclosed by the housing, the light source configured to emit germicidal light into the slot;
wherein the housing is configured to receive a wheel caster, at least partially, into the slot such that a wheel mounted to the caster is at least partially enclosed by the slot and the germicidal light is emitted onto the wheel.

20. The wheel disinfection assembly of claim 19, wherein the housing includes a caster interface on each side of the slot, the caster interfaces configured to snap fit to the caster to secure the wheel disinfection assembly to the caster.
